(19) United States
(12) Patent Application Publication
(10) Pub. No.: US 2021/0332407 A1
(43) Pub. Date: Oct. 28, 2021

(54) METHOD FOR MEASURING THE CONTAMINATION OF DRINKING WATER BY MICRO-ORGANISMS, IN A DRINKING WATER CONDUIT

(71) Applicant: BLUELAB WASSERANALYSESYSTEME GMBH, Tuebingen (DE)

(72) Inventor: Michael JAUSS, Tuebingen (DE)

(21) Appl. No.: 17/273,306

(22) PCT Filed: Aug. 27, 2019
(86) PCT No.: PCT/EP2019/072765

§ 371 Date: May 28, 2021

(30) Foreign Application Priority Data
Sep. 13, 2018 (DE) 10 2018 122 422.2

(51) Int. Cl. C12Q 1/06; G01N 35/00; C02F 1/44; C02F 1/00; E03B 7/07; E03C 1/10; G01N 33/18; F24D 17/00

(52) U.S. Cl. CPC C12Q 1/06; G01N 35/00009; G01N 35/0092; C02F 1/444; C02F 1/008; E03B 7/078; C02F 1/32; G01N 33/1826; F24D 17/0073; G01N 2035/00019; C02F 2209/02; C02F 2209/40; C02F 2303/04; E03C 1/10

(57) ABSTRACT

The invention is based on a method, particularly a locally fixed method, for measuring a contamination of drinking water by microorganisms (16) in a drinking water conduit (12).

It is proposed that a contamination of drinking water is predicted in at least one prediction procedure (88) by means of at least one contamination risk parameter, and an actual contamination of drinking water is measured in at least one measuring procedure (90).

### PRIOR ART

The invention relates to a method for measuring a contamination of drinking water by microorganisms in a drinking water conduit and an analysis device according to the preamble of claims 1 and 19.

There are already known methods for measuring a contamination of drinking water by microorganisms in a drinking water conduit.

The object of the invention is thus in particular to provide a common method and/or device with improved properties as relates to efficiency. The object is achieved according to the invention by means of the features of claims 1 and 19, whereas advantageous embodiments and refinements of the invention can be ascertained from the dependent claims.

### ADVANTAGES OF THE INVENTION

The invention relates to a method, in particular a locally fixed method, for measuring the contamination of drinking water by microorganisms, in a drinking water conduit.

It is proposed that, in at least one prediction procedure, a contamination of drinking water is predicted on the basis of at least one contamination risk parameter, and an actual contamination of drinking water is measured in at least one measuring procedure. This can hereby improve the efficiency of a method for measuring a contamination of drinking water. Further advantageously, a qualitative as well as a quantitative determination of the microorganisms can be implemented in the prediction procedure and/or measuring procedure. Particularly due to the combination of prediction procedure and measuring procedure, a validity of a determination of a contamination of drinking water can be improved. For example, a deviating result from the prediction procedure and the measuring procedure may indicate a faulty measurement. Especially advantageously, costs can be reduced, because a relevance of an implementation of the measuring procedure can be determined by the prediction procedure, and thus particularly an unnecessary measuring procedure can be avoided.

The method is particularly part of an operating program and is stored and/or can be executed particularly in a control unit. The term “control unit” here should particularly be understood as a unit with at least one processor unit and preferably at least one memory unit. The control unit particularly comprises at least the operating program which is configured for executing the method and particularly comprises at least one method sequence. The operating program can be executed with the processor unit and is preferably stored in the memory unit.

The term “drinking water conduit” here should particularly be understood as a conduit which is configured to convey drinking water, particularly at least hot water. For example, the drinking water conduit may be a conduit of a drinking water house connection, a drinking water house lead-in, a drinking water fixture, a drinking water faucet, a drinking water tank, particularly a drinking water tank heater, a drinking water circuit, or the like. In particular, a conduit of a drinking water fixture, a drinking water faucet, a drinking water tank, particularly a drinking water tank heater, or a drinking water circuit may be formed as a drinking water conduit conveying hot water. The term “hot water” in this case should particularly be understood as drinking water which has a water temperature of at least room temperature, particularly of 20° C.

Contamination by microorganisms in a drinking water conduit here should particularly be understood as a contamination of the drinking water conduit itself and/or of the drinking water conveyed by the drinking water conduit by microorganisms, particularly Legionella bacteria. The term “contamination” here should particularly be understood as a presence of microorganisms which exceeds a quantity of 10 microorganisms, preferably 50 microorganisms, and especially preferably 100 microorganisms per 100 mL drinking water. For example, according to the German Drinking Water Ordinance (TrinkwV 2001) for microorganisms, which are particularly Legionella bacteria in this case, a contamination of drinking water is present when a quantity of 100 microorganisms per 100 mL of drinking water is exceeded.

The term “procedure”, such as is used, for example, with the prediction procedure and/or the measuring procedure, should particularly be understood as at least one part of a method comprising at least one method step, preferably at least two method steps, and especially preferably several method steps. The term “prediction procedure” here should particularly be understood as a procedure in which a probability of a contamination is determined, particularly by means of the at least one contamination risk parameter, and particularly not by means of a direct measurement of the microorganisms causing the contamination of drinking water. In particular, the prediction procedure is implemented at least once, preferably at least twice, and especially preferably multiple times before at least one measuring procedure is implemented. In particular, at least one contamination risk parameter, which is used to predict the contamination of drinking water, is detected in the prediction procedure. The term “measuring procedure” here should particularly be understood as a procedure in which a contamination of drinking water by microorganisms is implemented directly by means of an at least quantitative and preferably qualitative measurement of the microorganisms. The term “contamination risk parameter” here should particularly be understood as a parameter which is at least correlated with a contamination of drinking water. A contamination of drinking water can preferably be at least predicted by means of the contamination risk parameter. The contamination risk parameter is particularly a flow volume, a temperature, a pH value, a salt content, a metal content, a degree of corrosion such as, for example, a corrosion decomposition product content, a biofilm content, particularly a thickness of a biofilm, or the like.

It is further proposed that the measuring procedure is carried out as a function of a predicted contamination of drinking water. Efficiency can advantageously be further improved. In particular, an unnecessary and/or faulty implementation of a measuring procedure can be avoided. In particular, the measuring procedure is implemented as a function of a probability of drinking water contamination determined in the prediction procedure. In particular, a frequency of the implementation of the measuring procedure is dependent on the probability of a contamination of drinking water.

It is further proposed that the contamination of drinking water is predicted in the prediction procedure by means of at least one further contamination risk parameter, which is different from the first contamination risk parameter. Efficiency can advantageously be further improved. In particular, the accuracy of the prediction procedure can be improved.

Furthermore, a contamination of drinking water could particularly be predicted in the prediction procedure by means of at least three, and especially preferably several different, contamination risk parameters. Furthermore, at least the further contamination risk parameter is detected in the prediction procedure.

It is further proposed that a temperature and/or a flow rate of the drinking water is considered as at least one contamination risk parameter in the production procedure. Efficiency can advantageously be especially improved. In particular, the accuracy of the prediction procedure can be improved due to the temperature and/or the flow rate, which can be determined relatively accurately.

It is further proposed that the prediction procedure is repeated. Efficiency can advantageously be further improved. In particular, the accuracy of a prediction can be improved by a repeated implementation. Especially advantageously, continual monitoring of the probability of a drinking water contamination can be achieved. In particular, the prediction procedure is implemented repeatedly after at least one predefined time interval. It is further conceivable that the prediction procedure is implemented with a predefined number of repetitions, particularly before the measuring procedure is carried out. Especially preferably, the prediction procedure is continually implemented.

It is further proposed that the prediction procedure comprises at least one method step in which the contamination of drinking water is predicted by means of a characteristic probability map. The efficiency of a prediction can advantageously be further improved. In particular, the prediction can be implemented in an especially simple way and manner. It is further conceivable that the contamination of drinking water is predicted by means of several characteristic probability maps, which are particularly based on various contamination risk parameters.

It is further proposed that the measuring procedure comprises at least one method step in which at least one test strip, which comprises at least one test field for implementing the measurement, is moved along a main extension direction of the test strip. Several test fields of the test strip for measuring the contamination of drinking water can advantageously be provided and/or replaced in an efficient way and manner. The term “test field” should particularly be understood as a section of the test strip which is configured for implementing a test, particularly a single test. Preferably, a test field is not reusable after use. In particular, the several test fields are arranged spaced apart from one another along the test strip. In particular, the test strip is moved in sections. Preferably, a movement of the test strip is paused at least sometimes, particularly between different method steps of the measuring procedure. The term “main extension direction” of an object here should particularly be understood as a direction which extends parallel to a longest edge of a smallest geometric cuboid which is still completely enclosing the object. A main extension of the test strip in this case should particularly be understood as an unrolled state of the test strip.

It is further proposed that the measuring procedure comprises at least one method step in which at least one test strip is moved along various test stations. Various individual steps of a test for measuring the contamination of drinking water can advantageously be implemented in an efficient way and manner. The test stations are particularly at least partially formed by the analysis device, a measuring device, and/or a cartridge device.

The term “test station” here should particularly be understood as a station at which at least one part for implementing the test is completed and along which the test strip is preferably moved during a test for the measurement. The term “analysis device” here should particularly be understood as a device which is configured at least for a prediction and/or at least for a measurement of a contamination of drinking water. The term “prediction” here should particularly be understood as an indicator of probability of a contamination of drinking water, which can be determined particularly by means of at least one contamination risk parameter, preferably without determining a presence and/or a quantity of microorganisms directly. The term “cartridge device” here should particularly be understood as at least one design and/or functional component and preferably a fully functional device which is configured for coupling to at least one measuring device for measuring a contamination of drinking water. The cartridge device is preferably formed to be replaceable. The term “replaceable” in this case should particularly be understood as replaceable with an at least substantially identical new cartridge device. In particular, the cartridge device can be coupled without tools. The phrase “at least substantially identical” should particularly be understood as identical to within production and/or assembly tolerances. The phrase “can be coupled” here should be understood as particularly non-positively and/or positively connectable. The phrase “non-positively and/or positively connected” in this case should particularly be understood as a detachable connection, in which a retaining force between two components is preferably transferred by means of a geometric interlocking of the components and/or a frictional force between the components.

The term “configured” here should particularly be understood as specially programmed, specially designed, and/or specially equipped. By the phrase that an object is configured for a certain function should particularly be understood that the object fulfills and/or executes this particular function in at least one application and/or operating state. It is further proposed that the measuring procedure comprises at least one method step in which at least one test strip is unrolled and/or rolled up, at least in sections. Efficiency can advantageously be achieved, particularly with respect to compactness, storage, and/or stowage of the test strip. The test strip is particularly unrolled from a supply spool and/or rolled onto a retractor spool.

It is further proposed that the measuring procedure comprises at least one method step in which a test field of a test strip is arranged to be covered at least by one further section of the test strip. The term “covered” here should particularly be understood as lying directly on top of one another, preferably adjacent one another. Efficiency can advantageously be achieved, particularly with respect to hygiene of the test strip, storage, and/or stowage of the test strip.

It is further proposed that the measuring procedure comprises at least one method step in which a drinking water sample of the drinking water is dispersed at a dispersion station. Efficiency can advantageously be advantageously improved. In particular, a measuring procedure, particularly the measuring accuracy, can be improved because microorganisms can be separated from one another due to the dispersion of the drinking water sample. In particular, agglomerates and aggregates of microorganisms can be separated from one another by the dispersion such that preferably individual microorganisms can be detected during a measurement. The dispersion station is a test station. The dispersion station comprises particularly at least one dispersion unit. The term “dispersion unit” should particularly be understood as a unit which is configured to break apart agglomerates and/or aggregates of microorganisms, which occur particularly with the formation of a biofilm, from one another into preferably individual microorganisms which are separate from one another.

It is further proposed that the measuring procedure comprises at least one method step in which a drinking water sample of the drinking water is microfiltered at a filter station by means of at least one test field of a test strip, in which the contaminating microorganisms are at least partially retained on the test field. The efficiency of a measurement can advantageously be further improved. In particular, a qualitative preselection can be made of the microorganisms to be measured. The filter station is particularly a test station.

In order to achieve an efficient quantitative determination of the contamination of drinking water particularly advantageously, it is further proposed that the measuring procedure comprises at least one method step in which microorganisms in the drinking water are provided with markers at a marker station. Preferably, the microorganisms retained in particular on the test field are provided with the markers. The term “markers” here should be particularly understood as dyes, particularly fluorescent dyes which are configured to couple to microorganisms, particularly a special type of microorganism, particularly Legionella bacteria, coliform bacteria, or the like. The marker station is particularly a test station. The marker station comprises at least one micro-metering unit which preferably comprises at least one micro-pump, which is configured for provision of the markers. The phrase “micro-metering unit” should particularly be understood as a unit which is configured for metering of a micro-dose. The term “micro-dose” should particularly be understood as a dose of a medium of less than 10 mL, preferably of less than 5 mL, and especially preferably of less than 2 mL. The marker station is particularly arranged along a main extension of the test strip, offset as relates to the filter station. In particular, the marker station is arranged directly next to the filter station.

It is further proposed that the markers intrinsically specify at least one microorganism type of the microorganisms. An intrinsic determination of the microorganisms can be achieved in an efficient way and manner. In particular, the markers are configured to mark a specific microorganism type of microorganism, particularly Legionella bacteria.

It is further proposed that the measuring procedure comprises at least one method step in which the excess markers are rinsed off at a rinse station. Preferably, at least a large portion of the excess markers is rinsed off at the filter station. In particular, the markers are rinsed off at the filter station by means of a liquid fluid, particularly by means of water. Preferably, any excess markers and/or excess fluid still present after a rinse process in the filter station is rinsed off at the rinse station. In particular, the markers are rinsed off of the test field at the rinse station with a fluid, such as air, particularly pressed through a filter of the test strip. The rinse station comprises at least one micro-metering unit which preferably comprises at least one micro-pump, which is configured for provision of the fluid used for rinsing. The rinse station is particularly arranged along a main extension of the test strip, offset as relates to the filter station. In particular, the rinse station is arranged directly next to the filter station. Preferably, the rinse station and the marker station form a common station.

It is further proposed that the measuring procedure comprises at least one method step in which microorganisms are optically detected at an optics station. Preferably, the optics station particularly comprises a fluorescent microscope. The optics station particularly comprises at least one optical sensor such as, for example, a camera sensor, particularly a CCD sensor. Furthermore, the optics station comprises at least one radiation source which is formed particularly as an LED and is preferably configured for the provision of UV light. The radiation source is particularly arranged in an incident light arrangement. A fluorescence of the markers excited by the radiation is detected particularly by the optical sensor of the optics station. Furthermore, the optics station has at least one optical filter. The optical filter is particularly arranged in a beam path of the optical sensor. The optical filter is configured to prevent, at least partially, preferably at least to a great extent, radiation in a spectral range of the radiation emitted by the radiation source. The phrase “at least to a great extent” in this case should be understood particularly as at least 55%, advantageously at least 65%, preferably at least 75%, especially preferably at least 85%, and especially advantageously at least 95%, and particularly however also completely. Furthermore, the optical filter is configured to permit radiation, particularly fluorescence, emitted by the markers by means of excitation due to the radiation, at least substantially undiminished. For example, the optical filter may be a dichroic filter, particularly in the form of a mirror. The optics station is particularly a test station. The optics station is particularly arranged next to, preferably directly next to, the rinse station and/or the marker station. The rinse station and/or the marker station is preferably arranged between the filter station and the optics station.

It is further proposed that the measuring procedure comprises at least one method step in which an image of the microorganisms is generated at the optics station with the resolution in a raster width of maximum 5 μm, preferably maximum 4 μm, and especially preferably maximum 3 μm. In particular, the optics station could record several images of the microorganisms and preferably average these for evaluation and/or generation of a main image. Furthermore, the image is image-processed, for example through stitching, depth sharpness, color saturation, sharpness, contrast increase, or the like.

It is further proposed that the measuring procedure comprises at least one method step in which a contamination of drinking water is determined at the optics station by means of a quantity of microorganisms. In particular, microorganisms provided particularly with markers and visible as individual points are counted in the image by means of an analysis algorithm, with the organisms preferably corresponding to at least one microorganism. Alternatively or additionally, it is conceivable that a diameter of the points is determined, particularly with the presence of aggregates and agglomerates of microorganisms, and a quantity of microorganisms contained therein is determined.

Further proposed is an analysis device for implementing the method. The analysis device preferably comprises a measuring device, which particularly at least partially and preferably at least to a great extent forms the filter station, the rinse station, the marker station, and/or the optics station. In particular, the analysis device comprises at least one control unit which is configured for implementing the method.

The method according to the invention and/or the analysis device according to the invention in this case should not be limited to the previously described application and embodiment. In particular, the method according to the invention and/or the analysis device according to the invention may have a number of individual elements, components, and units, as well as method steps which deviate from the number mentioned herein in order to fulfill a function described herein. In addition, the value ranges indicated in this disclosure should be considered disclosed and usable in any manner within the limit values.

### FIGURES

Further advantages result from the following description of figures. An exemplary embodiment of the invention is described in the figures. The figures, the description, and the claims contain numerous features in combination. One of ordinary skill in the art will expediently also consider the features individually and combine them into other reasonable combinations.

The following is shown:

FIG. 1 an analysis device in a schematic, perspective view;

FIG. 2 a portion of the analysis device having a sampling device, a measuring device, and a cartridge device in an exploded view;

FIG. 3 a portion of the analysis device with a sampling device;

FIG. 4 a diagram of an exemplary characteristic contamination map for predicting a contamination of drinking water;

FIG. 5 a portion of the cartridge device with a cartridge housing in a schematic, perspective view;

FIG. 6 a portion of the cartridge device without the cartridge housing in a schematic, perspective view;

FIG. 7 a portion of the cartridge device with a test strip in a schematic, perspective view;

FIG. 8 a portion of the cartridge device, the sampling device, and the measuring device in a schematic, perspective view; and

FIG. 9 a schematic flowchart of an exemplary method for operating the analysis device.

### DESCRIPTION OF THE EXEMPLARY EMBODIMENT

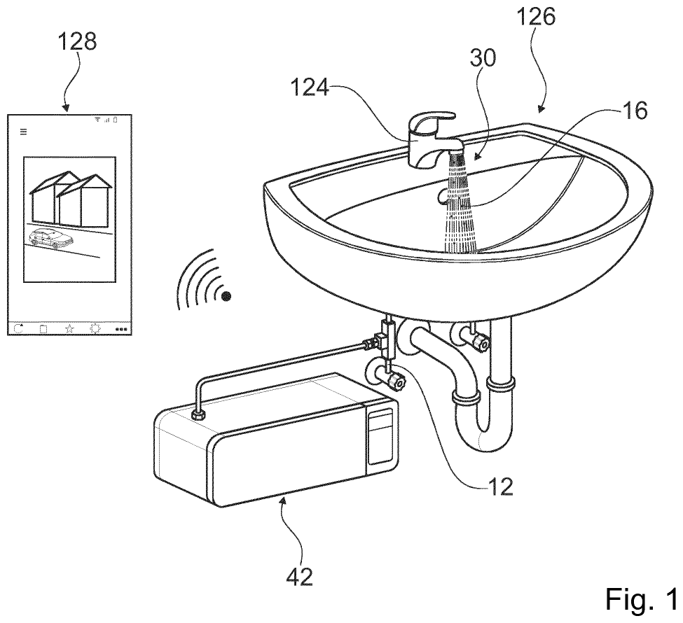

FIG. 1 shows a schematic, perspective view of an analysis device 42 and of a drinking water conduit 12. The analysis device 42 is assigned to a drinking water conduit 12. In the present case, the analysis device 42 is assigned locally fixed in position as relates to the drinking water conduit 12. The phrase “an object is assigned to at least one further object” should particularly be understood as the object is configured in at least one operating state and preferably durably in order to interact with the further object and is particularly arranged thereon. Furthermore, the term “fixed assignment” should be understood as the object is fixedly connected to the further object, particularly fluidically, and is particularly mounted thereon. The analysis device 42 is fixedly connected to the drinking water conduit 12. The analysis device 42 is configured for an analysis of a contamination by microorganisms 16, of the drinking water conduit 12. For the sake of clarity, the microorganisms 16 in FIG. 1 are shown in exaggerated form. Alternatively, the analysis device 42 may also be movable in order to assign it particularly to drinking water conduits 12 at various locations. It is further conceivable that the analysis device 42 can be assigned to several drinking water conduits 12. Preferably, the analysis device 42 is assigned to a final sampling point of a drinking water conduit system, i.e. particularly directly before reaching a final consumer.

The drinking water conduit 12 conveys drinking water 30. In the present case, the drinking water conduit 12 conveys hot water. The drinking water conduit 12 is a hot water conduit. The drinking water conduit 12 is connected to a faucet 124. The faucet 124 is part of a washbasin fixture 126. Alternatively, the drinking water conduit 12 may be a conduit, which conveys drinking water 30, of a drinking water house connection, a drinking water house lead-in, a drinking water tank, particularly a drinking water tank heater, or the like.

The analysis device 42 comprises at least one external unit 128. The external unit 128 is formed separately from the other components of the analysis device 42. The external unit 128 is provided with further components of the analysis device 42 in order to exchange data. The external unit 128 is configured at least for displaying analysis data. Furthermore, the external unit 128 is configured for control, particularly remote control, of the analysis device 42. In the present case, the external unit 128 is formed as a smart phone. Alternatively, the external unit 128 may also be formed as a different device, particularly a handheld unit, such as, for example, a tablet, a smart watch, a laptop, or the like. Furthermore, the external unit 128 could be a server, particularly a cloud server, such as, for example, a server from a central office.

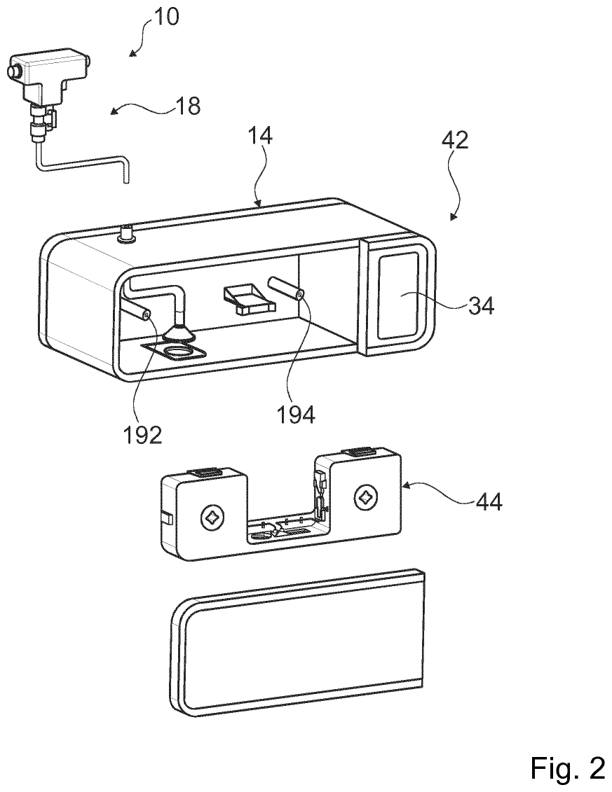

FIG. 2 shows a portion of the analysis device 42 in an exploded view. The analysis device 42 has at least one measuring device 14. Furthermore, the analysis device 42 has at least one sampling device 10. The term “sampling device” 10 should particularly be understood as at least one design and/or functional component and preferably a fully functional device which is configured for removing at least one drinking water sample from at least one of the drinking water conduits 12 particularly fixedly assigned to the sampling device 10. The sampling device 10 is configured to provide at least one drinking water sample to the measuring device 14. The sampling device 10 is arranged on the measuring device 14. Furthermore, the analysis device 42 has at least one cartridge device 44. The cartridge device 44 is arranged on the measuring device 14. The measuring device 14 comprises at least one mounting housing. The mounting housing is configured to accommodate the cartridge device 44 at least partially.

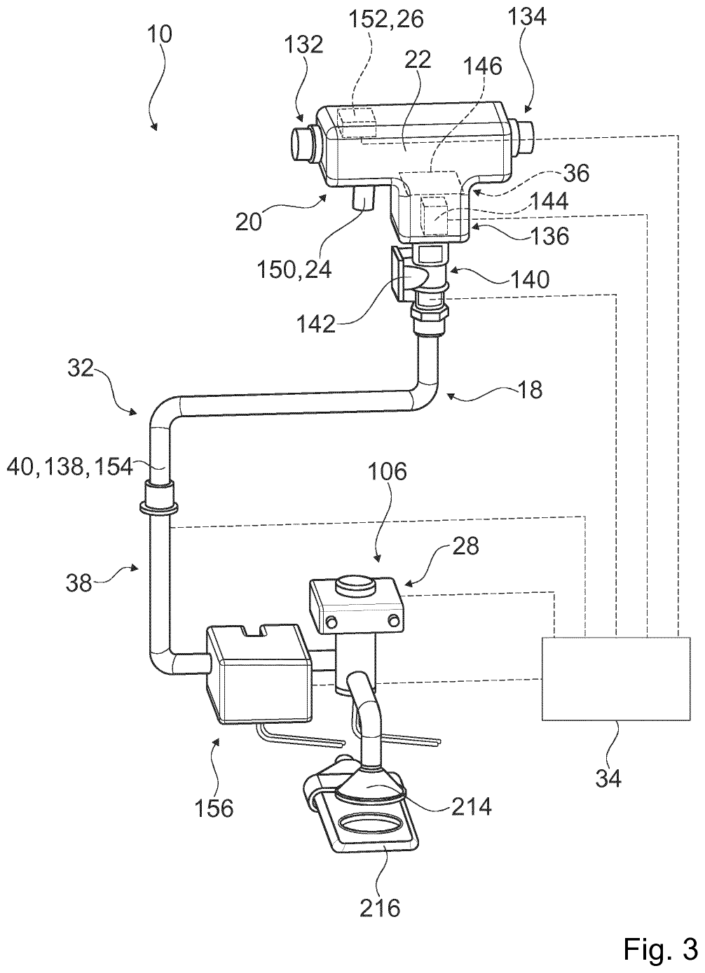

FIG. 3 shows a portion of the sampling device 10 in a schematic, perspective view. The sampling device 10 is configured at least for monitoring a predicted contamination of drinking water. In addition, the sampling device 10 is configured for removal of at least one drinking water sample from the drinking water conduit 12 (cf. FIG. 1). The sampling device 10 is assigned to the drinking water conduit 12. In the present case, the sampling device 10 is assigned locally fixed in position as relates to the drinking water conduit 12. The sampling device 10 is fixedly connected to the drinking water conduit 12.

The sampling device 10 has at least one extraction unit 18. The extraction unit 18 is configured for the extraction of drinking water. The extraction unit 18 is configured for removing at least a portion of a drinking water 30 conveyed by the drinking water conduit 12 as a drinking water sample from the drinking water conduit 12. In the present case, the extraction unit 18 forms a branch of the drinking water conduit 12. Furthermore, the extraction unit 18 may at least partially form the drinking water conduit 12 and/or a bypass of the drinking water conduit 12.

The extraction unit 18 has at least one connection 132. The connection 132 is configured as a drinking water inlet. The connection 132 is connected to the drinking water conduit 12. Furthermore, the extraction unit 18 has at least one additional connection 134. The additional connection 134 is configured as a drinking water outlet. The connection 132 and the additional connection 134 are arranged opposite one another fluidically.

Furthermore, the extraction unit 18 has at least one further connection 136. The further connection 136 is configured for extracting the drinking water. The further connection 136 is particularly arranged to be at least substantially transverse as relates to a primary flow direction of the drinking water 30 through the drinking water conduit 12. The phrase “at least substantially transverse” should particularly be understood as at least substantially different than parallel and preferably at least substantially perpendicular.

The phrase “at least substantially parallel” here should particularly be understood as an alignment of a direction relative to a reference direction, particularly in a plane, in which the direction and the reference direction form an angle of 0°, particularly with consideration of a maximum deviation of less than 8°, advantageously of less than 5°, and especially advantageously of less than 2°. The phrase “at least substantially perpendicular” here should particularly be understood as an alignment of a direction relative to a reference direction, particularly in a plane, in which the direction and the reference direction form an angle of 90°, particularly with consideration of a maximum deviation of less than 8°, advantageously of less than 5°, and especially advantageously of less than 2°.

The extraction unit 18 has at least one connection head 22. The connection head 22 is formed as a three-way connection head. The connection head 22 is formed in a T-shape. The connection head 22 is configured for connecting to the drinking water conduit 12. The connection head 22 provides the connection 132. Furthermore, the connection head 22 provides the additional connection 134. In addition, the connection head 22 provides the further connection 136.

The sampling device 10 has at least one sensor unit 20. The sensor unit 20 is configured for detecting at least one contamination risk parameter. The contamination risk parameter is a temperature of the drinking water 30. In the present case, the sensor unit 20 is configured for detecting at least one further contamination risk parameter. The further contamination risk parameter is different from the first contamination risk parameter. The further contamination risk parameter is a flow rate of the drinking water 30.

The sensor unit 20 is arranged at least partially in the extraction unit 18. The phrase “sensor unit 20 is arranged at least partially in the extraction unit 18” should particularly be understood as at least one sensor 150, 152 of the sensor unit 20 and preferably all sensors 150, 152 of the sensor unit 20 are arranged in the extraction unit 18. Furthermore, the phrase to be “arranged in something” should actually also be understood as somewhat integrated. The sensor unit 20 has at least one sensor 150. The sensor 150 is configured for detecting the contamination risk parameter. The sensor 150 is arranged in the connection head 22. The sensor 150 is assigned to the connection 132. The sensor 150 is arranged in the region of the connection 132. In the present case, the sensor 150 is formed as a temperature sensor 24.

The sensor unit 20 has at least one further sensor 152. The further sensor 152 is configured for detecting the further contamination risk parameter. The further sensor 152 is arranged in the connection head 22. The further sensor 152 is assigned to the connection 132. The further sensor 152 is arranged in the region of the connection 132. In the present case, the further sensor 152 is formed as a flow rate sensor 26.

The extraction unit 18 has at least one valve unit 140. The valve unit 140 is configured for the targeted extraction of drinking water. The valve unit 140 in this case is at least partially integrated into the connection head 22. Furthermore, the valve unit 140 is at least partially arranged on the connection head 22, particularly at the further connection 136.

The valve unit 140 comprises at least one valve 142. In the present case, the valve 142 is formed as a switching valve. The valve 142 can be opened and/or closed for a targeted sample extraction by switching. In addition, the valve 142 is formed as a metering valve. The valve 142 is configured for setting a sampling quantity of a drinking water sample. The valve unit 142 is arranged on the connection head 22, particularly at the further connection 136.

The valve unit 140 has at least one further valve 144. The further valve 144 is formed as a one-way valve. The further valve 144 is integrated into the connection head 22. The further valve 144 is assigned to the further connection 136. The valve unit 140 may comprise a quantity of valves 142, 144 which deviates from the one shown here. It would be conceivable that the valve unit 140 comprises only one valve 142, 144 which combines the functions of a metering valve, a one-way valve, and/or a switching valve. Furthermore, the valve unit 140 could also be completely integrated into the connection head 22.

The extraction unit 18 has at least one extraction line 138. The extraction line 138 connects the drinking water conduit 12 to a measuring device 14 of the analysis device 42 (cf. FIG. 2). The extraction line 138 is connected to the further connection 136 fluidically. The valve unit 140 is arranged between the connection head 22 and the extraction line 138. The extraction line 138 is formed as a hose. Furthermore, the extraction line 138 is at least partially flexible. The extraction line 138 is at least partially formed from a flexible material such as, for example, a plastic, particularly rubber. It is conceivable that the extraction unit 18 could be formed, at least partially, as a single piece with the connection head 22 and/or the valve unit 140. For example, the valve unit 140 could be integrated into the extraction line 138. The phrase that a first object and a second object are formed “at least partially as a single piece” should particularly be understood as at least one element and/or a portion of the first object and at least one element and/or portion of the second object are formed as a single piece. The term “single piece” should particularly be understood as connected with at least a firm bond, for example by means of a welding process, an adhesive process, an injection molding process, and/or a different process that appears to be reasonable to one skilled in the art, and/or advantageously molded into one piece such as, for example, due to production from a casting and/or due to production in a single-component or multi-component injection molding process and advantageously comprising a single blank. The phrase “connected with a firm bond” should be understood as parts by mass which are held together through atomic or molecular forces such as, for example, when soldering, welding, bonding, and/or curing.

The sampling device 10 comprises at least one filter unit 36. The filter unit 36 is configured for macro-filtration of the drinking water 30. The term “macro-filtration” should particularly be understood as filtration with a pore size of maximum 1000 μm, preferably of maximum 100 μm, and especially preferably of maximum 10 μm. The filter unit 36 is arranged at least partially in the extraction unit 18. The filter unit 36 is fluidically arranged upstream of the extraction line 138. Furthermore, the filter unit 36 is fluidically arranged upstream of the valve unit 140. The filter unit 36 comprises at least one filter element 146. The filter element 146 is replaceable. The filter element 146 can be, for example, a filtering screen which is particularly in the form of an aerator. The filter element 146 is arranged in the connection head 22. The filter element 146 is arranged in the region of the connection 136. The filter element 146 has a pore size of maximum 1000 μm. Furthermore, the filter unit 36 may comprise two or more filter elements 146 which are particularly formed to be different from one another. For example, the filter unit 36 could comprise filter elements 146 of various pore sizes. The filter unit 36 could also be arranged as a portion of the valve unit 140 or be arranged between the connection head 22, valve unit 140, and/or the extraction line 138.

The sampling device 10 comprises at least one disinfection unit 38. The disinfection unit 38 is configured to eliminate or kill off the microorganisms 16. The disinfection unit 38 is configured for disinfecting at least the extraction unit 18. By means of the disinfection unit 38, the extraction unit 18 can be disinfected between measurements of a contamination of drinking water in order to prevent distortion of a measurement due to microorganisms 16 accumulating and/or proliferating in the extraction unit 18.

In the present case, the disinfection unit 38 is configured for physical disinfection. The disinfection unit 38 is configured to heat the extraction unit 18 to at least 70° C. The disinfection unit 38 comprises at least one temperature-control element 40. The extraction line 138 forms the disinfection unit 38. The extraction line 138 is heatable, particularly being formed as a temperature-control element 40. Alternatively or additionally, it is conceivable that the disinfection unit 38 has a disinfection element which is separate from the extraction line 138. For example, the disinfection element could be a heating coil. It is further conceivable that the disinfection element may be formed as a Peltier element.

Alternatively or additionally, the disinfection unit 38 could be configured for physical disinfection through radiation with electromagnetic radiation such as, for example, UV light. In this case, the disinfection element could be formed as a UV light source. It would be preferable in this case for the extraction line 138 to be formed at least permeable to UV light. It is conceivable that the disinfection unit 38 could be configured for chemical disinfection. The term “chemical disinfection” should particularly be understood as disinfection by means of an oxidizing agent such as, for example, chlorine, chlorine oxide, hydrogen peroxide, dimethyl dicarbonate, silver ions, and/or ozone. In particular, a disinfection element, which is configured for chemical disinfection and/or for disinfection with UV light, could be arranged between the connection head 22 and the extraction line 138.

The sampling device 10 has at least one dispersion unit 28. The dispersion unit 28 is configured for dispersing microorganisms 16 in drinking water 30. The dispersion unit 28 is configured to break apart agglomerates and/or aggregates of microorganisms 16, which occur particularly with the formation of a biofilm, from one another into preferably individual microorganisms 16 which are separate from one another. In the present case, the dispersion unit 28 is formed as an ultrasound dispersion unit. The dispersion unit 28 disperses microorganisms 16 in the drinking water 30 by introducing ultrasound into the extraction unit 18. To this end, the dispersion unit 28 provides ultrasound at a frequency of at least 16 kHz, preferably of at least 18 kHz, especially preferably of at least 20 kHz, and very especially preferably of at least 30 kHz. The dispersion unit 28 is arranged on the extraction unit 18. The dispersion unit 28 is connected at one end of the extraction line 138.

The sampling device 10 has at least one further sensor unit 156. The further sensor unit 156 is configured for detecting a flow rate for metering a drinking water sample through the extraction unit 18, particularly the extraction line 138. The further sensor unit 156 is formed as an ultrasound flow meter.

The sampling device 10 comprises at least one decoupling unit 32. The decoupling unit 32 is configured for vibratory decoupling of the vibrations propagating mechanically along the extraction unit 18. The decoupling unit 32 is configured for decoupling the dispersion unit 28 in a vibratory-mechanical manner. The decoupling unit 32 is configured for decoupling the sensor unit 20 in an at least partially vibratory-mechanical manner. The decoupling unit 32 is configured for decoupling the dispersion unit 28 and the sensor unit 20 from each other in an at least partially vibratory-mechanical manner. The decoupling unit 32 is configured for thermally decoupling the sensor unit 20 from the remaining components of the sampling device 10, particularly from the dispersion unit 28. The decoupling unit 32 has at least one decoupling element 154 for the decoupling.

In the present case, the decoupling unit 32 and the extraction unit 18 are formed at least partially as a single piece. In the present case, the decoupling unit 32 is formed at least partially by the extraction line 138. The extraction line 138 forms the decoupling element 154.

The extraction unit 18 of the sampling device 10 is fluidically connected to a drinking water sample connection 214 of the measuring device 14. The drinking water sample connection 214 is connected, some distance away, to a drinking water sample outlet 216 of the measuring device 14.

The analysis device 42 has at least one control unit 34. The control unit 34 is part of the sampling device 10. The control unit 34 is configured for the control of further components of the analysis device 42, particularly of the sampling device 10, the measuring device 14, and/or the cartridge device 44. Furthermore, the control unit 34 has a communicating connection to the external unit 128. The control unit 34 has a wireless communicating connection to the external unit 128, for example via radio, WLAN, Bluetooth, GSM, or the like. The control unit 34 provides data to the external unit 128. The control unit 34 can be actuated by the external unit 128. In the present case, the control unit 34 is part of the measuring device 14 (cf. FIG. 2).

The control unit 34 comprises at least one processor unit (not shown). Furthermore, the control unit 34 comprises at least one memory unit (not shown). The processor unit and the memory unit are not shown in the figures for the sake of clarity. The control unit 34 further comprises at least one operating program. The operating program can be executed in the processor unit. Furthermore, the operating program is stored in the memory unit. The operating program is configured for executing a method for operating the analysis device 42, particularly the sampling device 10, the measuring device 14, and/or the cartridge device 44.

The control unit 34 is configured for predicting a contamination of drinking water at least by means of the contamination risk parameter. In the present case, the control unit 34 is configured to also predict the contamination of drinking water by means of the further contamination risk parameter. Preferably, the control unit 34 uses a characteristic contamination map 158. The characteristic contamination map 158 is stored in the control unit 34, particularly the memory unit of the control unit 34. The characteristic contamination map 158 is a characteristic map in which at least the values of at least the contamination risk parameters and the further contamination risk parameter are plotted opposite each other. Furthermore, the contamination risk parameters are assigned a probability of a drinking water contamination in the characteristic contamination map 158. It is further conceivable that the control unit 34 determines a probability of a contamination of drinking water by means of a characteristic contamination curve, which comprises at least values of one contamination risk parameter, which are assigned a respective probability of a drinking water contamination.

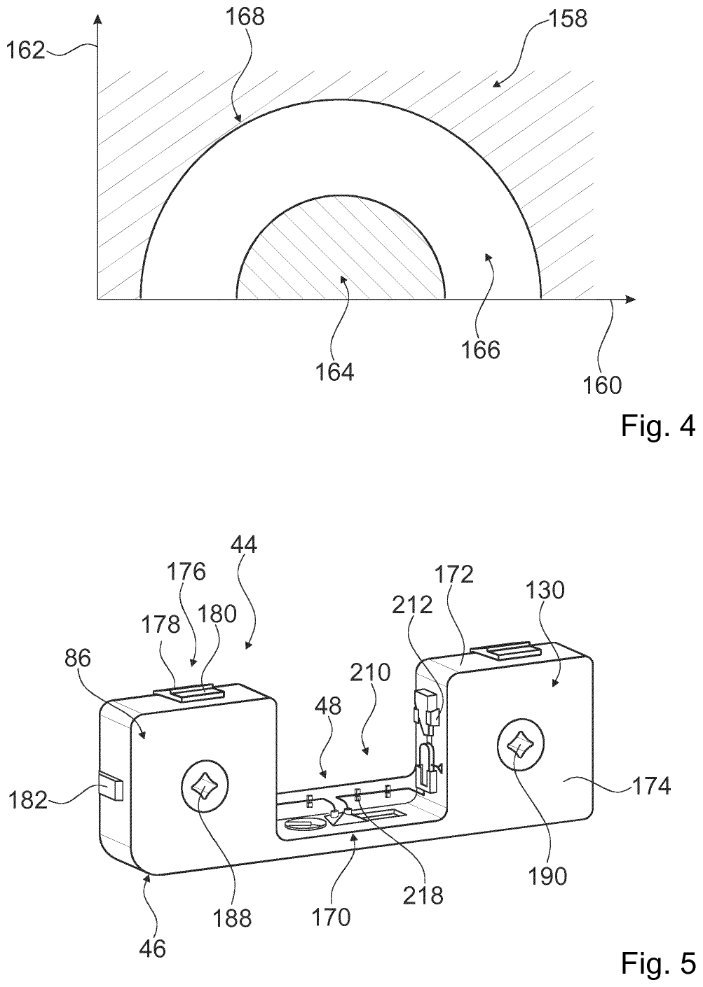

FIG. 4 shows a diagram of an exemplary characteristic contamination map 158. The diagram has an x-axis 160. The contamination risk parameter is plotted on the x-axis 160. Furthermore, the diagram has a y-axis 162. The further contamination risk parameter is plotted on the x-axis 162. In the present case, the contamination risk parameter and the further contamination risk parameter are linked to a respective probability of a drinking water contamination by means of comparison values stored in the control unit 34. Furthermore, they can be linked together by means of a mathematical equation such as, for example, an equation of ellipse and/or of circle. The characteristic contamination map 158 has at least different probability ranges 164, 166, 168 of a contamination of drinking water. The different probability ranges 164, 166, 168 shown in FIG. 4 should be considered particularly only an example, particularly the probabilities and/or value ranges of the probability ranges 164, 166, 168. In particular, the different probability ranges 164, 166, 168 may have probabilities and/or value ranges which deviate from the probabilities and/or value ranges indicated by the embodiment shown in FIG. 4.

The characteristic contamination map 158 has a probability range 164. A contamination of drinking water is predicted in the probability range 164 with an exemplary probability of at least 85%. The probability range 164 in the present case is in an exemplary value range of the contamination risk parameter of at least 27° C. and maximum 60° C. In the present case, the probability range 164 with an exemplary value range of the further contamination risk parameter is at least 0% and a maximum 45% of a maximum flow rate of drinking water 30 through the drinking water conduit 12.

The characteristic contamination map 158 has a further probability range 166. A contamination of drinking water is predicted in the further probability range 166 with an exemplary probability of at least 50%. The probability of a drinking water contamination of the further probability range 166 is less than the probability of a drinking water contamination of probability range 164. In the present case, the further probability range 166 with an exemplary value of the contamination risk parameter is at least 20° C. and maximum 65° C., particularly minus the value range of probability range 164. In the present case, the further probability range 166 with an exemplary value range of the further contamination risk parameter is at least 0% and a maximum 66% of a maximum flow rate of drinking water 30 through the drinking water conduit 12, particularly minus the value range of probability range 164.

The characteristic contamination map 158 has an additional probability range 168 of a contamination of drinking water. A contamination of drinking water is predicted in the additional probability range 168 with an exemplary probability of at least 30%. The probability of a drinking water contamination of the additional probability range 168 is less than the probability of a drinking water contamination of further probability range 166. In the present case, the additional probability range 168 with an exemplary value of the contamination risk parameter is at least 0° C. and maximum 100° C., particularly intersecting with the value ranges of further probability range 166. In the present case, the additional probability range 168 with an exemplary value range of the further contamination risk parameter is at least 0% and a maximum 100% of a maximum flow rate of drinking water 30 through the drinking water conduit 12, particularly intersecting with the value ranges of further probability range 166.

The probabilities of a contamination of the respective probability ranges 164, 166, 168 are discretized in the present case. There is a differentiation among three different discrete probabilities. Alternatively, it is conceivable that a probability can be determined continually by means of the values of the contamination risk parameters.

To this end, the control unit 34 is configured for implementing sampling as a function of at least one predicted contamination of drinking water. In the present case, the control unit 34 implements the sampling as a function of a probability, determined by means of the contamination risk parameters, of drinking water contamination of the respective probability ranges 164, 166, 168. The control unit 34 implements a frequency of sampling as a function of the predicted contamination of drinking water, particularly of a probability of the drinking water contamination. In this case, a respective frequency of sampling depends on the probability of contamination of drinking water advantageously with weighting with a factor of a maximum sampling frequency. For example, the control unit 34 implements sampling more frequently with a probability of drinking water contamination of probability range 164 than with a probability of drinking water contamination of probability range 166. Furthermore, the control unit 34 implements sampling more frequently with a probability of drinking water contamination of further probability range 166 than with a probability of drinking water contamination of additional probability range 168. In this case, a frequency of sampling with a probability of drinking water contamination of the additional probability range 168 corresponds to a frequency of at least once in three years, particularly in accordance with the German Drinking Water Ordinance (TrinkwV 2001). Alternatively or additionally, it is conceivable that the control unit 34 can automatically implement sampling when the probability of contamination of drinking water exceeds a stored reference value.

FIG. 5 shows a portion of the cartridge device 44 in a schematic, perspective view. The cartridge device 44 is replaceable. The cartridge device 44 is configured for the measuring device 14 for measuring a contamination of the drinking water by microorganisms 16, in the drinking water conduit 12. The cartridge device 44 has at least one cartridge housing 46. The cartridge housing 46 is formed as a type of cassette. The cartridge housing 46 has a spectacle-like shape. The cartridge housing 46 has at least one storage section 86. Furthermore, the cartridge housing 46 has at least one further storage section 130. Storage section 86 and the further storage section 130 are at least substantially mirror images of one another. The cartridge housing 46 has a bar 170. The bar 170 connects storage section 86 and further storage section 130 to one another.

The cartridge housing 46 has a reservoir 172. The reservoir 172 is configured for arranging further components of the cartridge device 44. Furthermore, the cartridge housing 46 has a cover 174. The cover 174 closes the reservoir 172.

The cartridge housing 46 can be coupled to the measuring device 14 (cf. FIG. 4). The cartridge device 44 has at least one coupling unit 176 for the coupling. In the present case, the coupling unit 176 is formed as a quick coupler. The term “quick coupler” should particularly be understood as a preferably mechanical and/or magnetic unit, which is configured for coupling at least two components to one another, preferably with one hand, particularly very especially advantageously with a single hand movement of an operator, without tools, without destruction, and/or repeatedly. The coupling unit 176 has at least one latching element 178. The latching element 178 is arranged on the cartridge housing 46, particularly the reservoir 172. In the present case, the latching element 178 is formed with the cartridge housing 46, particularly the reservoir 172, as a single piece. The latching element 178 is arranged in the region of the storage section 86. The latching element 178 is configured for establishing a positive and/or non-positive connection to a corresponding latching element (not shown) of the measuring device 14. Furthermore, the coupling unit 176 has a further latching element. The further latching element is formed to be at least substantially identical to latching element 178. The further latching element is arranged in the region of the further storage section 130. Furthermore, the coupling unit 176 has at least one actuating element 180. The actuating element 180 is configured for suspending a coupling between the cartridge housing 46 and the measuring device 14. In the present case, the actuating element 180 is formed as a lever. The actuating element 180 is connected to latching element 178 as a single piece. Furthermore, the coupling unit 176 has a further actuating element. The further actuating element is formed to be at least substantially identical to actuating element. The further actuating element is arranged in the region of further storage section 130.

In addition, the coupling unit 176 has at least one guide element 182. The guide element 182 is configured for guiding the cartridge housing 46 during coupling to the measuring device 14. The guide element 182 is arranged on the cartridge housing 46, particularly the reservoir 172. In the present case, the guide element 182 is formed with the cartridge housing 46, particularly the reservoir 172, as a single piece. The guide element 182 is arranged in the region of storage section 86. Furthermore, the coupling unit 176 has a further guide element. The further guide element is formed to be at least substantially identical to guide element 182. The further guide element is arranged in the region of further storage section 130. Alternatively or in addition to the embodiment shown by example in FIG. 5, it is conceivable that the coupling unit 176 has at least one screw, at least one welded connection, or the like for coupling the cartridge housing 46 to the measuring device 14.

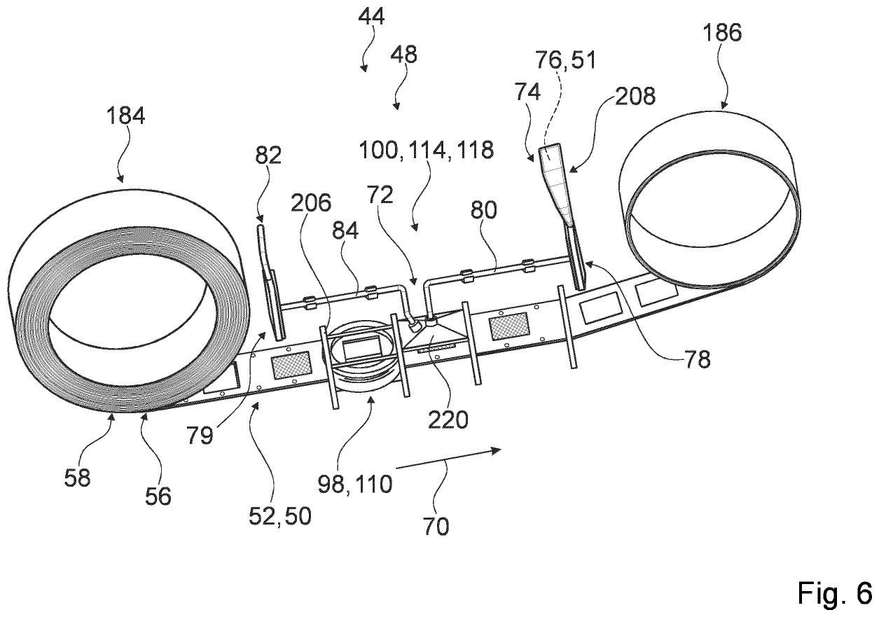

Furthermore, the cartridge device 44 has at least one test unit 48. The term “test unit” 98, 100, 102 should particularly be understood as a unit which contributes to the provision of test setups at least for implementing a measurement of the contamination of drinking water. The test unit 48 is at least partially arranged in the cartridge housing 46. The test unit 48 has at least one consumable material 50. The consumable material 50 is configured for implementing at least one test for measuring the contamination of drinking water. Furthermore, the test unit 48 has at least one further consumable material 51 (cf. FIG. 6). The term “consumable material” 50, 51 should particularly be understood as a material which must be disposed of after use with one test and/or cannot be used for a further test.

FIG. 6 shows a portion of the cartridge device 44 without the cartridge housing 46 in a schematic, perspective view. The test unit 48 has at least one test strip 52. At least the test strip 52 is a consumable material 50 of the test unit 48.

In at least one stored state of the test strip 52, the test strip 52 is stored rolled up, at least in sections. Preferably, the test unit 48 has at least one supply spool 184. The supply spool 184 is arranged in the storage section 86 (cf. FIG. 5). In the stored state, the test strip 52 is at least partially rolled onto the supply spool 184. In order to carry out a test for measuring a contamination of drinking water, the test strip 52 can be unrolled from the supply spool 184. Due to the rolled-up storage, at least one test strip section 56 of the test strip 52 at least partially covers at least one further test strip section 58 of the test strip 52 in the stored state of the test strip 52.

Furthermore, the test unit 48 has at least one retractor spool 186. The retractor spool 186 is arranged in the further storage section 130. In the stored state, the test strip 52 is at least partially rolled onto the retractor spool 186. After completion of a test for measuring a contamination of drinking water, the test strip 52 can be rolled onto the retractor spool 186. Due to the rolled-up storage, at least one test strip section 56 of the test strip 52 at least partially covers at least one further test strip section 58 of the test strip 52 in the stored state of the test strip 52.

Due to the simultaneous roll-off and roll-on of the test strip 52, it is moved along its main extension direction 70 in the unrolled state.

In order to roll the test strip 52 on and/or off, the retractor spool 186 and the supply spool 184 each have a shaft mount 188, 190. The shaft mounts 188, 190 are rotationally symmetrical. The shaft mounts 188, 190 each have a cross-shaped cross-section. The shaft mounts 188, 190 are each configured for receiving a drive shaft 192, 194. The measuring device 14 in this case comprises two correspondingly formed drive shafts 192, 194 (cf. FIG. 2).

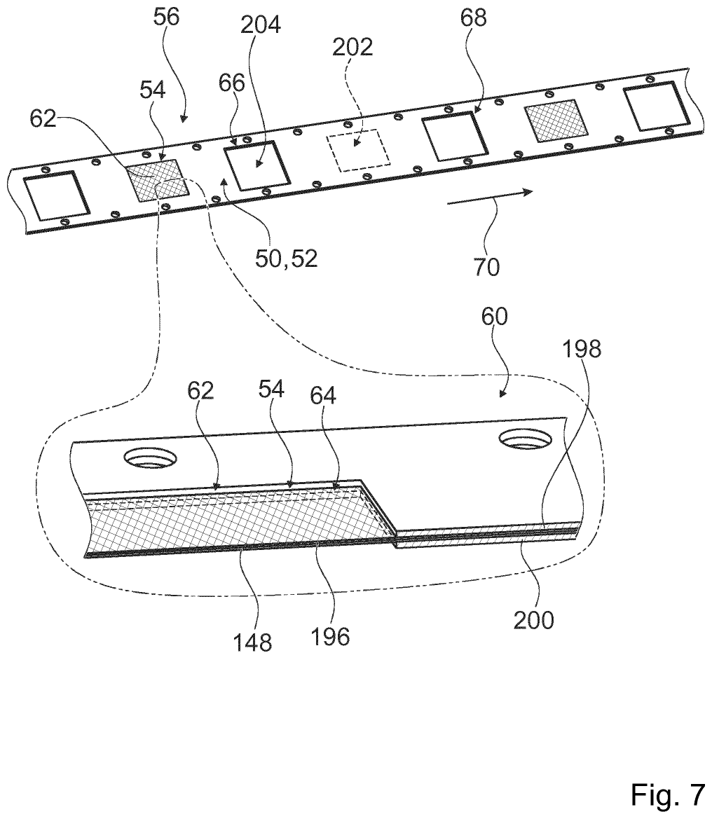

FIG. 7 shows a portion of the test strip 52 in the unrolled state of the test strip section 56, in a schematic, perspective view.

The test strip 52 has a layer structure 60. The term “layer structure” 60 should particularly be understood as a structure with at least two layers, which are formed different from each other. The test strip 52 has at least one filter layer 196. The filter layer 196 is configured for filtering a drinking water sample of drinking water during a measurement. The filter layer 196 has a pore diameter of maximum 0.45 μm. Alternatively, it is conceivable that the filter layer 196 has a pore diameter of maximum 0.2 μm. The filter layer 196 particularly has a layer thickness of maximum 0.1 mm. The filter layer 196 is formed by a nonwoven filter material. The filter layer 196 consists at least partially of a hygroscopic material. The filter layer 196 is free of any hydrophilic material.

Furthermore, the test strip 52 has at least one carrier layer 198. The carrier layer 198 is configured for supporting the filter layer 196. The carrier layer 198 has a layer thickness of maximum 0.5 mm. The carrier layer 198 consists at least partially of a plastic such as, for example, polyethylene and/or polypropylene. The carrier layer 198 has a smooth surface. The carrier layer 198 is connected to the filter layer 196 with a firm bond. In the present case, the carrier layer 198 is welded to the filter layer 196. The carrier layer 198 is welded to the filter layer 196 by means of a calendering process. Alternatively, the layers could also be bonded together with adhesive.

Furthermore, the test strip 52 has at least one further carrier layer 200. The further carrier layer 200 is formed to be at least substantially identical to carrier layer 198. Alternatively, the further carrier layer 200 could also be formed, however, differently from carrier layer 198. The filter layer 196 is enclosed between the carrier layers 198, 200. Accordingly, the layer structure 60 in the present case is in the form of a sandwich construction. The term “sandwich construction” should particularly be understood as a layer structure 60 with at least three layers, which are preferably at least partially formed differently from one another, in which at least one layer is enclosed between two carrier layers 198, 200 which are preferably formed to be at least substantially identical. The filter layer 196 is welded to the carrier layer 198 and to a support layer 148 of the test strip 52. The support layer 148 is arranged between the carrier layers 198, 200. The support layer 148 is configured for stabilizing the filter layer 196. The support layer 148 is formed as a, particularly coarse-meshed, cloth. The support layer 148 has a layer thickness which is at least substantially similar to the layer thickness of the filter layer 196.

The test strip 52 comprises at least one test field 54. The test field 54 comprises at least one filter 62. The filter 62 is configured for filtering microorganisms 16 out of the drinking water 30. In the present case, the filter 62 is formed by the filter layer 196 of the test strip 52.

The test strip 54 has a depression 64. The depression 64 is configured for receiving microorganisms 16 from the drinking water sample. The depression 64 is formed by removal in areas, particularly punching out, the carrier layer 198, 200 of the test strip 52 in the region of the test field 54. The depression 64 exposes the filter layer 196 arranged on the carrier layer 198. In this manner, the filter layer 196 forms the filter 62 of the test field 54 in the region of the test field 54. By flushing the filters 62 with the drinking water sample, particularly microorganisms 16 which collect in the depression 64 are retained on the filter 62.

Furthermore, the test strip 52 has at least one cleaning field 66. The cleaning field 66 is configured for rinsing with drinking water 30. The cleaning field 66 has a larger surface area than the test field 54. The cleaning field 66 has a larger extension transversely as relates to a main extension direction 70 of the test strip 52 than the test field 54. Furthermore, it is proposed that the cleaning field 66 has at least one recess 204. The recess 204 is a complete recess. The recess 204 relates to all layers from which the test strip 52 is composed. The carrier layer 198, the further carrier layer 200, and the filter layer 196 are removed and particularly punched out in the region of the cleaning field 66. In the region of the cleaning field 66, the filter layer 196 is removed along a complete extension of the test strip 52, transversely as relates to the main extension direction 70 of the test strip 52. Due to the recess 204 of the cleaning field 66, test field 54 is separated, using fluid transport, particularly using capillary forces, from further test fields of the test strip 52 along test field 52.

The test field 54 and the cleaning field 66 are arranged offset with respect to one another along the main extension direction 70 of the test strip 52. Furthermore, a separating field 202 is arranged between the cleaning field 66 and a further cleaning field 68. The test field 54 and the cleaning field 66 are arranged alternately with respect to one another in the main extension direction 70 of the test strip 52.

Furthermore, the test strip 52 has at least one separating field 202. The separating field 202 is configured to keep the cleaning field 66 and the further cleaning field 68 apart from one another. Furthermore, the separating field 202 is configured for covering the test field 54 in the stored state. The separating field 202 is marked by a dashed line in FIG. 7. The separating field 202 has a full layer structure 60 of the test strip 52 and is formed free of depressions or recesses.

The test field 54, the separating field 202, and the cleaning field 66 in this case are arranged in a particularly repeating test strip sequence in the main extension direction 70 of the test strip 52. The test strip 52 comprises several test strip sequences arranged directly next to each other. One test strip sequence comprises one test field 54, one cleaning field 66, one separating field 202, and one further cleaning field 68.

The cartridge device 44 has at least one sealing unit 72 (cf. FIG. 6). The sealing unit 72 seals off the test unit 48 at least in sections. The sealing unit 72 is arranged in a seal with at least one test strip section 56 of the test strip 52. The sealing unit 72 separates different test stations 98, 100, 102 of the analysis device 42 from each other with a fluid-tight seal. The sealing unit 72 seals off an exposed test strip section 56 of the test strip 52. The sealing unit 72 has at least one seal 206. The seal 206 is formed as a sealing lip. The seal 206 has direct contact with the test strip 52, in which particularly only the depression 64 of the test field 54 is not in contact with the seal 206.

Furthermore, the sealing unit 72 has at least one further seal. The further seal is formed to be at least substantially identical to seal 206. In the present case, the sealing unit 72 has a total of four seals 206. Two seals 206 seal off each of the test stations 98, 100, 102 in a fluid-tight manner. For the sake of clarity, only one seal 206 is provided with a reference numeral in the figures. Seal 206 and the further seal are arranged offset with respect to one another along the main extension direction 70 of the test strip 52. In the present case, the sealing unit 72 has four seals 206.

The test unit 48 comprises at least one supply unit 74. The supply unit 74 is configured for the provision of markers 76 for marking the microorganisms 16. The markers 76 are further consumable material 51. The supply unit 74 is at least partially arranged on the cartridge housing 46. In the present case, the supply unit 74 is arranged on an outer side of the cartridge housing 46. The supply unit 74 is arranged in the region of further storage section 130. Alternatively, it is also conceivable that the supply unit 74 could be arranged at least partially within the cartridge housing 46.

The supply unit 74 particularly has a memory unit 208. The markers 76 are stored in the memory unit 208. The markers 76 are consumable material 51 which can be replaced or refilled. For example, the memory unit 208 may be replaceable with a new memory unit filled with new markers. Furthermore, it is conceivable that the memory unit 208 can be refilled with new markers.

The supply unit 74 comprises at least one micro-metering unit 78. The micro-metering unit 78 is fluidically connected to the memory unit 208. The micro-metering unit 78 is configured for conveying the markers 76. The micro-metering unit 78 comprises at least one micro-pump (not shown further here).

Furthermore, the supply unit 74 has at least one supply line 80. The supply line 80 is fluidically connected to a marker station 114 of the measuring device 14. The supply line 80 is configured for supplying the markers 76 to the test strip 52, particularly the test field 54. The supply unit 74, particularly the micro-metering unit 78, can be controlled by the control unit 34 for metering the markers 76.

The cartridge device 44 has at least one placement unit 210 (cf. FIG. 5). The placement unit 210 is configured for placing the supply unit 74 on the cartridge housing 46. The placement unit 210 comprises at least one placement element 212. The placement element 212 is configured for placement of the memory unit 208. The placement element 212 is formed as a receiving pocket. The memory unit 208 can be placed in the placement element 212, particularly it can be inserted. The placement element 212 is formed with the cartridge housing 46 as a single piece. Furthermore, the placement unit 210 has at least one further placement element. For the sake of clarity, only one placement element 212 has been provided with a reference numeral. The further placement element is configured for placement of the micro-metering unit 78. The further placement element is formed as a receiving pocket. The further placement element is formed with the cartridge device 44 as a single piece. In addition, the placement unit 210 has at least one additional placement element 218. The additional placement element 218 is configured for placement of the supply line 80. The additional placement element 218 is formed as a receiving bracket. The additional placement element 218 is arranged in the region of a bar 170.

The test unit 48 has at least one rinsing unit 82. The rinsing unit 82 is configured for rinsing at least one consumable material 50 of the test unit 48. The rinsing unit 82 comprises at least one further micro-metering unit 79. The further micro-metering unit 79 is formed to be at least substantially identical to micro-metering unit 78. Furthermore, the rinsing unit 82 is arranged at least partially in the region of the storage section 86.

The rinsing unit 82 is arranged on an outer side of the cartridge housing 46.

The further micro-metering unit 79 is fluidically connected to ambient air. The further micro-metering unit 79 is configured for generating an air flow. Furthermore, the rinsing unit 82 has one further supply line 84. The further supply line 84 is fluidically connected to the further micro-metering unit 79. The further supply line 84 is fluidically connected to a rinse station 118 of the measuring device 14.

Furthermore, the cartridge device 44 has a supply connection 220. The supply connection 220 at least partially forms the rinse station 118 and/or the marker station 114. The supply connection 220 is arranged on an unrolled test strip section 56 of the test strip 52. The supply connection 220 is arranged on an exposed test strip section 56 of the test strip 52. The supply connection 220 is formed as a funnel. The supply connection 220 is connected to the supply unit 74 and/or the rinsing unit 82, particularly via the respective supply lines 80, 84. The supply connection 220 is arranged in the region of the bar 170. The further supply line 84 is arranged offset next to the supply connection 220 along the main extension direction 70 of the test strip 52.

The measuring device 14 comprises several test stations 98, 100, 102. The test strip 52 is moved along the test stations 98, 100, 102. The measuring device 14 comprises at least one dispersion station 106. Furthermore, the measuring device 14 comprises the filter station 110. Furthermore, the measuring device 14 comprises the marker station 114. Furthermore, the measuring device 14 comprises the rinse station 118. Furthermore, the measuring device 14 comprises an optics station 122. The rinse station 118 is arranged offset as relates to the filter station 110 in the main extension direction 70. The marker station 114 is arranged offset as relates to the filter station 110 in the main extension direction 70. The marker station 114 and the rinse station 118 are at least partially formed as a single piece. The optics station 122 is arranged offset as relates to the marker station 114 in the main extension direction 70. The rinse station 118 and the marker station 114 are arranged between the filter station 110 and the optics station 122.

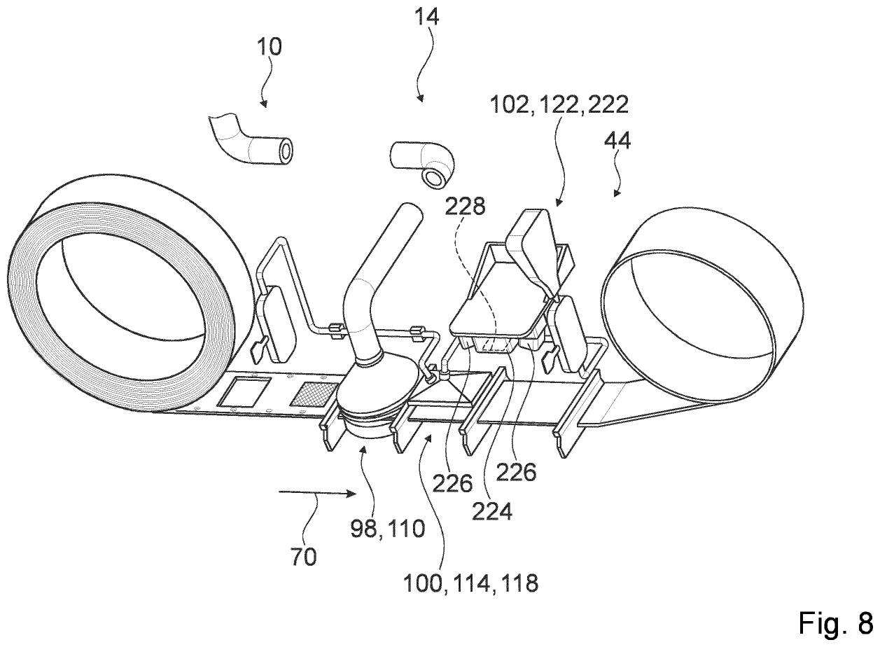

FIG. 8 shows a portion of the measuring device 14 together with the parts of the cartridge device 44 and the sampling device 10. The measuring device 14 is configured for measurement of a drinking water contamination by microorganisms 16, in the drinking water conduit 12. The measuring device 14 is assigned to the drinking water conduit 12. In the present case, the measuring device 14 is assigned locally fixed in position as relates to the drinking water conduit 12. The measuring device 14 is fixedly connected to the drinking water conduit 12. In the present case, the sampling device 10 connects the measuring device 14 to the drinking water conduit 12. The measuring device 14 is configured for using a drinking water sample provided by the sampling device 10 for the measurement of the contamination of drinking water. The measuring device 14 comprises an optics station 122. The optics station 122 is configured for a measurement of the contamination of drinking water.

The measuring device 14 comprises at least one measuring unit 222. The measuring unit 222 at least partially forms the optics station 122. The measuring unit 222 comprises at least one optical sensor 224. In the present case, the optical sensor 224 is formed as a camera sensor, particularly as a CCD sensor. Furthermore, the measuring unit 222 comprises at least one radiation source 226. In the present case, the measuring unit 222 has two radiation sources 226. Furthermore, the radiation sources 226 are formed as LEDs. The radiation sources 226 are configured for the provision of UV light. The radiation sources 226 and the optical sensor 224 are arranged such that an incident light arrangement results.

A fluorescence of the markers 76 triggered by the radiation from the radiation sources 226 is detected by the optical sensor 224 of the measuring unit 222. Furthermore, the measuring unit 222 has at least one optical filter 228. The optical filter 228 is arranged in a beam path of the optical sensor 224. The optical filter 228 is configured to prevent, at least partially, preferably at least to a great extent, radiation in a spectral range of the radiation emitted by the radiation sources 226. Furthermore, the optical filter 228 is configured to permit radiation, particularly fluorescence, emitted from the markers 76 due to excitation from the radiation, at least substantially undiminished. The optical filter 228 is formed as a dichroic filter 228.

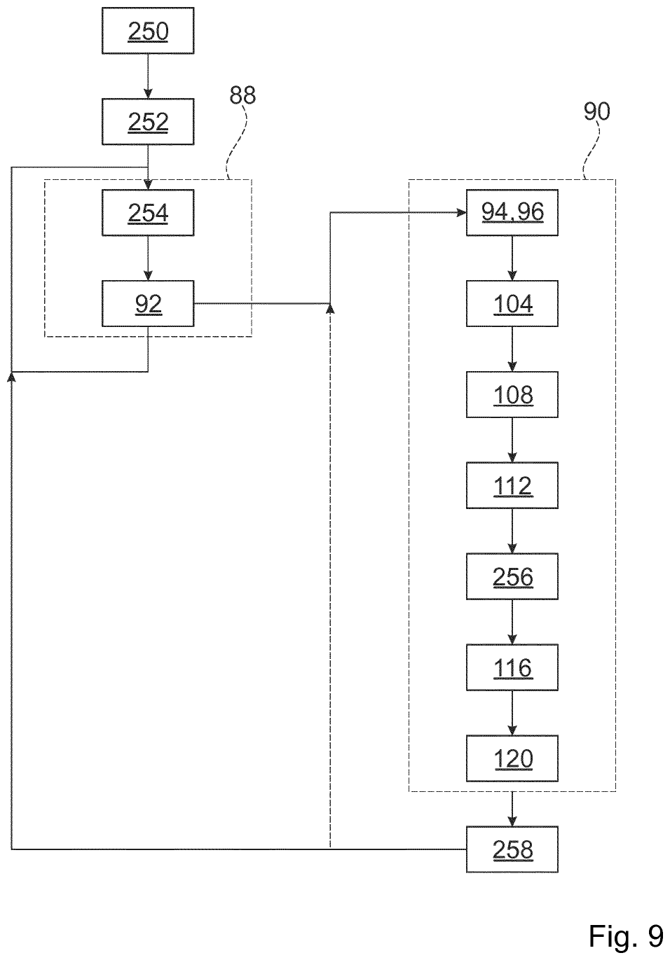

A schematic flowchart of a method for operating at least the analysis device 42 is shown in FIG. 9. The method is part of the operating program which is executed by the control unit 34. The method is a method for predicting and measuring a contamination of drinking water by microorganisms 16, in the drinking water conduit 12. In the present case, the method is implemented in a locally fixed position, i.e. at a location of a drinking water conduit 12 fixedly assigned to the analysis device 42. Alternatively, the method could also be implemented at a location different from the drinking water conduit 12, particularly after sampling carried out separately from the method.

The method for operating the analysis device 42 corresponds at least substantially to a method, particularly in a locally fixed position, for measuring a contamination of drinking water by microorganisms 16, in a drinking water conduit 12. With the method, particularly the locally fixed method, for measuring a contamination of drinking water by microorganisms 16 in a drinking water conduit 12, a contamination of drinking water is predicted in at least one prediction procedure 88 by means of at least one contamination risk parameter, and an actual contamination of drinking water is measured in at least one measuring procedure 90.

The method comprises a method step 250 in this case. The cartridge device 44 is inserted into the measuring device 14 in the method step 250. The cartridge housing 46 of the cartridge device 44 is coupled to the measuring device 14. In the event that the cartridge device 44 is already coupled to the measuring device 14, method step 250 can be omitted.

The method comprises at least one further method step 252. In further method step 252, an analysis of the contamination of drinking water is initiated by the analysis device 42. In the present case, the analysis is initiated by the external unit 128. The control unit 34 of the analysis device 42 in this case is controlled by the external unit 128, particularly remote-controlled. If an analysis is initiated autonomously locally fixed on the analysis device 42 and/or if an automatic initiation is provided such as, for example, based on a scheduler, the external unit 128 can be omitted. Furthermore, this method step 252 can also be omitted if there is continual operation of the analysis device 42.

The method comprises at least one prediction procedure 88. The prediction procedure 88 is configured for predicting the contamination of drinking water. The contamination of drinking water is predicted in the prediction procedure 88. A contamination of drinking water is predicted in the prediction procedure 88 by means of at least one contamination risk parameter. A temperature of the drinking water 30 is considered a contamination risk parameter. In the present case, the contamination of drinking water is predicted in the prediction procedure 88 by means of at least one further contamination risk parameter. The further contamination risk parameter is different from the first contamination risk parameter. A flow rate of the drinking water 30 is considered as at least the one further contamination risk parameter. The prediction procedure 88 is carried out repeatedly, particularly continually.

The prediction procedure 88 comprises at least one method step 254. In method step 254, at least one contamination risk parameter is detected by the sensor unit 20. The sensor unit 20 detects the contamination risk parameter. In the present case, the sensor 24, 150 detects the contamination risk parameter. Furthermore, the sensor unit 20 detects at least the further contamination risk parameter. In the present case, the further sensor 26, 152 of the sensor unit 20 detects the further contamination risk parameter.

The prediction procedure 88 comprises at least one further method step 92. In further method step 92, the contamination of drinking water is predicted by means of the characteristic contamination map 158. In the event that a reference value is exceeded due to a predicted probability of the contamination of drinking water, a measuring procedure 90 is initiated. In the event that the predicted probability of the contamination of drinking water falls below the reference value, the prediction procedure 88 is repeated.

The method comprises at least one measuring procedure 90. The measuring procedure 90 is configured for measuring the contamination of drinking water. In measuring procedure 90, an actual contamination of drinking water is measured. The measuring procedure 90 is implemented after at least one implementation of the prediction procedure 88. The measuring procedure 90 is implemented as a function of a predicted contamination of drinking water.

The measuring procedure 90 comprises at least one method step 94. In method step 94, the test strip 52 is moved along a main extension direction 70 of the test strip 52. The test strip 52 is unrolled, at least in sections. Simultaneously, the test strip 52 is rolled up. In a stored state of the test strip 52, a test field 54 of the test strip 52 is arranged so as to be covered by a further test strip section 58 of the test strip 52. If the test strip 52 is moved, the test strip section 58 of the test strip 52, which was previously covering the test field 54, is removed therefrom.

The measuring procedure 90 comprises at least one further method step 96. In method step 96, the test strip 52 is moved along various test stations 98, 100, 102. Initially, the test strip 52 is moved such that a test field 54 of the test strip 52 is arranged in the region of the filter station 110. Method step 96 takes place at least partially at the same time as method step 94.

The measuring procedure 90 comprises at least one further method step 104. In method step 104, a drinking water sample of the drinking water 30, which was removed by the sampling device 10, is dispersed at a dispersion station 106.

The measuring procedure 90 comprises at least one further method step 108. In further method step 108, the drinking water sample of the drinking water 30 is micro-filtrated at a filter station 110 by means of at least one test field 54 of a test strip 52. The contaminating microorganisms 16 are at least partially retained on the test field 54.

The measuring procedure 90 comprises at least one further method step 112. In further method step 112, the test strip 52 is moved further in the main extension direction 70 until it is positioned in the region of the marker station 114. In further method step 112, microorganisms 16 are provided with markers 76 at the marker station 114. The markers 76 intrinsically specify at least one microorganism type of the microorganisms 16.

Furthermore, the measuring procedure 90 comprises at least one further method step 256. In further method step 256, the filter station 110 is cleaned. A cleaning field 66 is positioned in the region of the filter station 110 through movement of the test strip 52. This occurs automatically due to the arrangement of the cleaning field 66 and of the test field 54 as relates to one another as well as the filter station 110 and the marker station 114 when the test field 54 is positioned in the region of the marker station 114. The filter station 110 is cleaned by rinsing with drinking water 30.

The measuring procedure 90 comprises at least one further method step 116. In further method step 116, excess markers 76 are flushed out at the rinsing station 118. This occurs due to the ambient air provided by the rinsing unit 82. At the rinsing station 118, excess markers 76 which were not flushed out and/or excess fluid are flushed out at the filter station 110 by means of a liquid fluid, particularly by means of water. In the present case, the marker station 114 and the rinsing station 118 are positioned identically such that the test strip 52 does not have to be moved further to carry out method step 116.

The measuring procedure 90 comprises at least one further method step 120. In further method step 120, the microorganisms 16 are optically detected at an optics station 122. The detection is implemented by the measuring unit 222. In this case, an image of the microorganisms 16 is created. The image in this case has a resolution of a raster width of maximum 5 μm. Furthermore, a contamination of drinking water is determined in further method step 120 at the optics station 122 by means of a quantity of the microorganisms 16. Preferably, individual points on the image are analyzed and quantitatively counted by means of an algorithm. In the event that a contamination of drinking water is determined, a user can be informed thereof, for example via the external unit 128.

Furthermore, the method comprises a further method step 258. In further method step 258, the extraction unit 18 is disinfected by the disinfection unit 38. Subsequently, the prediction procedure 88 can be repeated. Alternatively or additionally, the measuring procedure 90 could also be repeated, particularly in the event that a measurement of the contamination deviates significantly from a predicted contamination.

With respect to further method steps of the method for operating the analysis device 42 and/or the method for measuring a contamination of drinking water by microorganisms 16 in a drinking water conduit 12, the preceding description of the analysis device 42 can be referenced, because this description is similar as well to the method for operating the analysis device 42 and/or to the method for measuring a contamination of drinking water by microorganisms 16 in a drinking water conduit 12, and thus all features with respect to the analysis device 42 are considered to be disclosed also in reference to the method for operating the analysis device 42 and/or the method for measuring a contamination of drinking water by microorganisms 16 in a drinking water conduit 12.

### REFERENCE NUMERALS

10 Sampling device

12 Drinking water conduit

14 Measuring device

16 Microorganism

18 Extraction unit

20 Sensor unit

22 Connection head

24 Temperature sensor

26 Flow rate sensor

28 Dispersion unit

30 Drinking water

32 Decoupling unit

34 Control unit

36 Filter unit

38 Disinfection unit

40 Temperature-control element

42 Analysis device

44 Cartridge device

46 Cartridge housing

48 Test unit

50 Consumable material

51 Consumable material

52 Test strip

54 Test field

56 Test strip section

58 Test strip section

60 Layer structure

62 Filter

64 Depression

66 Cleaning field

68 Cleaning field

70 Main extension direction

72 Sealing unit

74 Supply unit

76 Marker

78 Micro-metering unit

79 Micro-metering unit

80 Supply line

82 Rinsing unit

84 Supply line

86 Storage section

88 Prediction procedure

90 Measuring procedure

92 Method step

94 Method step

96 Method step

98 Test station

100 Test station

102 Test station

104 Method step

106 Dispersion station

108 Method step

110 Filter station

112 Method step

114 Marker station

116 Method step

118 Rinse station

120 Method step

122 Optics station

124 Faucet

126 Washbasin fixture

128 External unit

130 Storage section

132 Connection

134 Connection

136 Connection

138 Extraction line

140 Valve unit

142 Valve

144 Valve

146 Filter element

148 Support layer

150 Sensor

152 Sensor

154 Decoupling element

156 Sensor unit

158 Characteristic contamination map

160 x-axis

162 y-axis

164 Probability range

166 Probability range

168 Probability range

170 Bar

172 Reservoir

174 Cover

176 Coupling unit

178 Latching element

180 Actuating element

182 Guide element

184 Supply spool

186 Retractor spool

188 Shaft mount

190 Shaft mount

192 Drive shaft

194 Drive shaft

196 Filter layer

198 Carrier layer

200 Carrier layer

202 Separating field

204 Recess

206 Seal

208 Memory unit

210 Placement unit

212 Placement element

214 Drinking water sample connection

216 Drinking water sample outlet

218 Placement element

220 Supply connection

222 Measuring unit

224 Optical sensor

226 Radiation source

228 Optical filter

250 Method step

252 Method step

254 Method step

256 Method step

258 Method step

I claim:

1. A method, particularly a locally fixed method, for measuring a contamination of drinking water by microorganisms in a drinking water conduit, wherein a contamination of drinking water is predicted in at least one prediction procedure by means of at least one contamination risk parameter, and an actual contamination of drinking water is measured in at least one measuring procedure.

2. The method according to claim 1, wherein the measuring procedure is implemented as a function of a predicted contamination of drinking water.

3. The method according to claim 1, wherein the contamination of drinking water is predicted in the prediction procedure by means of at least one further contamination risk parameter, which is different from the first contamination risk parameter.

4. The method according to claim 1, wherein a temperature and/or a flow rate of the drinking water is considered as at least one contamination risk parameter in the prediction procedure.

5. The method according to claim 1, wherein the prediction procedure is implemented repeatedly.

6. The method according to claim 1, wherein the prediction procedure comprises at least one method step wherein the contamination of drinking water is predicted by means of a characteristic probability map.

7. The method according to claim 1, wherein the measuring procedure comprises at least one method step wherein at least one test strip, which comprises at least one test field for implementing the measurement, is moved along a main extension direction of the test strip.

8. The method according to claim 1, wherein the measuring procedure comprises at least one method step wherein at least one test strip is moved along various test stations.

9. The method according to claim 1, wherein the measuring procedure comprises at least one method step wherein at least one test strip is unrolled and/or rolled up, at least in sections.

10. The method according to claim 1, wherein the measuring procedure comprises at least one method step wherein a test field of a test strip is arranged covered by a further test strip section of the test strip.

11. The method according to claim 1, wherein the measuring procedure comprises at least one method step wherein a drinking water sample of the drinking water is dispersed at a dispersion station.

12. The method according to claim 1, wherein the measuring procedure comprises at least one method step wherein a drinking water sample of the drinking water is micro-filtrated at a filter station by means of at least one test field of a test strip, wherein the contaminating microorganisms are at least partially retained on the test field.

13. The method according to claim 1, wherein the measuring procedure comprises at least one method step wherein microorganisms of the drinking water are provided with markers at a marker station.

14. The method according to claim 13, wherein the markers intrinsically specify at least one microorganism type of the microorganisms.

15. The method according to claim 13, wherein the measuring procedure comprises at least one method step wherein excess markers are flushed out at a rinsing station.

16. The method according to claim 1, wherein the measuring procedure comprises at least one method step wherein microorganisms are optically detected at an optics station.

17. The method according to claim 16, wherein the measuring procedure comprises at least one method step wherein an image of the microorganisms is created with a resolution of a raster width of maximum 5 μm at the optics station.

18. The method according to claim 16, wherein the measuring procedure comprises at least one method step wherein a contamination of drinking water is determined at the optics station by means of a quantity of the microorganisms.

19. An analysis device which is configured for implementing the method according to claim 1.
